(19) United States
(12) Patent Application Publication
(10) Pub. No.: US 2018/0110465 A1
(43) Pub. Date: Apr. 26, 2018

(54) METHODS AND SYSTEMS FOR PHYSIOLOGIC MONITORING

(71) Applicant: Reza NAIMA, San Francisco, CA (US)

(72) Inventor: Reza NAIMA, San Francisco, CA (US)

(21) Appl. No.: 15/791,346

(22) Filed: Oct. 23, 2017

Related U.S. Application Data
(60) Provisional application No. 62/411,482, filed on Oct. 21, 2016.

(51) Int. Cl. A61B 5/00; A61B 5/0205

(52) U.S. Cl. CPC A61B 5/681; A61B 5/0205; A61B 5/04085; A61B 2562/0219; A61B 5/0006

(57) ABSTRACT

Devices and methods including wearable monitoring devices for a user's wrist that may be configured to intelligently and automatically decide when to monitor a user without requiring direct user input.

### CROSS REFERENCE TO RELATED APPLICATIONS

This patent application claims priority to U.S. Provisional Patent Application No. 62/411,482, titled “METHODS AND SYSTEMS FOR PHYSIOLOGIC MONITORING” and filed on Oct. 21, 2016.

This patent application may be related to application Ser. No. 14/958,915, filed on Dec. 3, 2015 and titled “METHODS AND SYSTEMS FOR DETECTING PHYSIOLOGY FOR MONITORING CARDIAC HEALTH.”

Each of these applications is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

Wearable physiological monitoring apparatuses that may operate without user interaction while providing robust monitoring, particularly for automatically determining the times to perform measurements allowing for the highest level of signal quality while minimizing the number of required measurements per day.

### BACKGROUND

Being able to measure physiologic data in a non-invasive way that yields to high user compliance is critical in order to ensure continuous data is collected and acted upon for numerous health monitoring applications. Although there are many types of devices which can be used for collecting non-invasive data, there are shortcomings to many such systems. For example, many patches exist which can be applied to the torso enabling measurement of numerous physiologic parameters such as heart rate, respiration rate, blood oxygen saturation, activity, etc. However, requiring an individual to wear an adhesive patch (either disposable, or reusable) for an extended period of time on the torso can lead to skin irritation, is uncomfortable, and will result in low compliance.

Other systems require direct user interaction—these include blood pressure cuffs or a simple weight scale. Repetitive daily interaction can lead to lower compliance by virtue of the individual forgetting to interact with the device or fatigue from performing the same activity repetitively.

There are a number of additional factors which can lead to a reduction in compliance. For wearable devices, these include
short battery life which requires frequent recharging of the device
uncomfortable form factor
skin irritation
complex interface requiring user interaction
difficulty removing and putting the device back on
hard to read interface
requires using the device in conjunction with a smartphone or personal computer

All these issues are further complicated if the subject is elderly, unfamiliar with technology, has difficulty with dexterous mobility, suffers from dementia, has difficulty seeing, amongst other reasons.

For these reasons, it's critical that a deployed noninvasive monitoring technology be simple to use, comfortable, easy to put on and take off, require no daily interaction, and at most very infrequent recharging of the battery in order to achieve high compliance—without which no amount of technical ability to monitor is sufficient.

### SUMMARY OF THE DISCLOSURE

The present invention relates to method and apparatuses for preventing and detecting (e.g., predicting) Heart failure. Heart failure affects 1 in 5 people in the U.S., resulting in a decreased quality of life and the largest cost to the Medicare system. The symptoms of heart failure come on gradually and by the time they are noticed, it is often too late for preventative care resulting in costly hospitalizations. The physiologic changes leading to worsening heart failure and hospitalization are well understood. The technology described herein continuously monitors these parameters and relays them to the caregiver providing up to three weeks advance notice of worsening conditions. This allows for proactive care, preventing unnecessary hospitalizations, and increasing quality of life for the patients while saving billions of dollars to the healthcare system.

Described herein are apparatuses, including devices and systems, such as wearable electronics (e.g., configured as a wrist band, arm bank, ankle band, etc.) that can pair with a smartphone (or other wearable electronics device), e.g., over Bluetooth, to provide remote telemetry or use WiFi to directly stream data to the cloud for further analysis. The data may relayed directly to an Electronic Medical Records (EMR) system of the caregiver allowing for streamlined access without altering the current workflow of the caregiver.

Since heart failure is most prevalent in the elderly population, the device may be optimized for use by the elderly; for example, the device may include no display, no interface, and no adjustments required for use. In some variations, the device simply slips onto the wrist (or other appendage). Wireless telemetry may allow the device to detect when the device is not being used or if there is a problem, enabling corrective actions. And when the battery is low, the entire device may light up, notifying the patient in a simple manner.

The data collected (and may modify the collection time/amount) by one or more predictive algorithms that may determine the ideal times to perform measurements allowing for the highest level of signal quality while minimizing the number of required measurements per day. This allows for months of battery life without requiring the device to be recharged and making the device easier to use.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 is an example of an apparatus, configured as a wristband, as described herein.

FIG. 2 is an example of a strap of a wristband.

FIG. 3 is another example of an apparatus as described herein.

FIG. 4 is an example of an apparatus having a pre-biased band, as described herein.

FIGS. 5A-5D illustrate views of another example of an apparatus as described herein.

FIG. 6 is an example of a graph showing user activity.

FIG. 7 is an example of an omnidirectional tilt and vibration sensor that may be used with any of the devices described herein.

FIG. 8 is an example of a circuit that may be used as part of the control circuitry for the apparatuses described herein.

### DETAILED DESCRIPTION

There are certain strategies that can be deployed which can yield high compliance. These may include the form factor.

### A. Wrist-Worn

In order to achieve high compliance, a form factor that is comfortable must be deployed. Anything adhesive, bulky, or placed in an uncomfortable spot reduces user compliance. The ideal location for such a system would be the wrist as numerous physiologic signals can be measured at this location, and most people are used to and comfortable with a watch-like or band-like form factor. This is especially true for the elderly populations who have grown up wearing watches.

### B. Skin Contact

Additionally, the device must be easy to put on and take off, yet leave little to no room for adjustment as proper skin/electrode or skin/optical contact is required for good quality data acquisition. If the device is too loose, then improper skin contact can result in bad data acquisition. Conversely, if the device is too tight then the device can become uncomfortable causing the individual to remove the device resulting in low compliance.

For any type of interface (e.g. electrical, optical, pressure), the ideal range of pressures must be empirically determined. The minimum pressure which gives consistently good data will be the ideal pressure to be used in the system. Once the proper level of pressure is determined (“Ideal Pressure”), the band must be able to consistency deliver this pressure. Two methods are presented below:

### Full Band

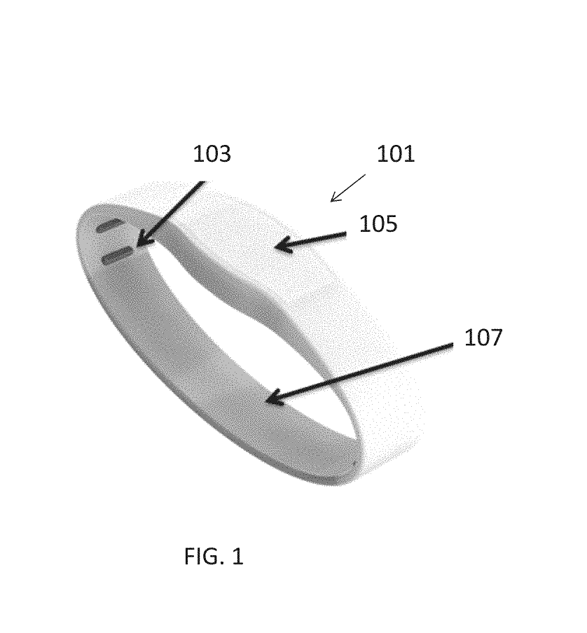

The band must be designed to allow it to be stretched over the fist, and it will exhibit a fairly flat strain/stress response for a given range of wrist diameters. The stress will result in the application of the Ideal Pressure to the wrist, and will be as constant as possible over a range of wrist diameters. A number of different band sizes will be used to accommodate a range of band sizes. See, e.g., FIG. 1. FIG. 1, and accompanying text, describes one example of a wearable device (wrist device) as described herein. Any of the features described may be absent or modified.

In FIG. 1, the device 101 may include a PCB that is either a rigid flex design where there will be traces that come down the side of the apparatus (e.g., “watch” or band) and terminate with metal electrodes 103 This may have the benefit of allowing the mounting of buffer amps next to the sense electrodes. Though running wires down the sides may be used instead. The electronics may be enclosed in a plastic shell 105 then over molded with a material (e.g., plastic) that is not very elastic (stretchy). In some variations, the apparatus may include bottom portion of the watch 107 (that may not include any electronics) that may be made of a stretchy (e.g., elastic) material such as a plastic or other polymer. This may allow it to stretch over the wearer's fist and apply the correct pressure to the wrist for the sensor to work (contact the skin reliably) without applying too much pressure to be uncomfortable. Ideally, this may be accomplished by using a material that has a relatively flat strain/stress curve, wherein it may apply the same stress independent of the wrist diameter.

In this version, the top portion will remain the same size and different lengths of the blue portion will be made and joined (e.g. over molded, fused, glued) together to allow for different sizes. The right choice of materials will allow for fewer sizes to accommodate a larger number of wrist diameters.

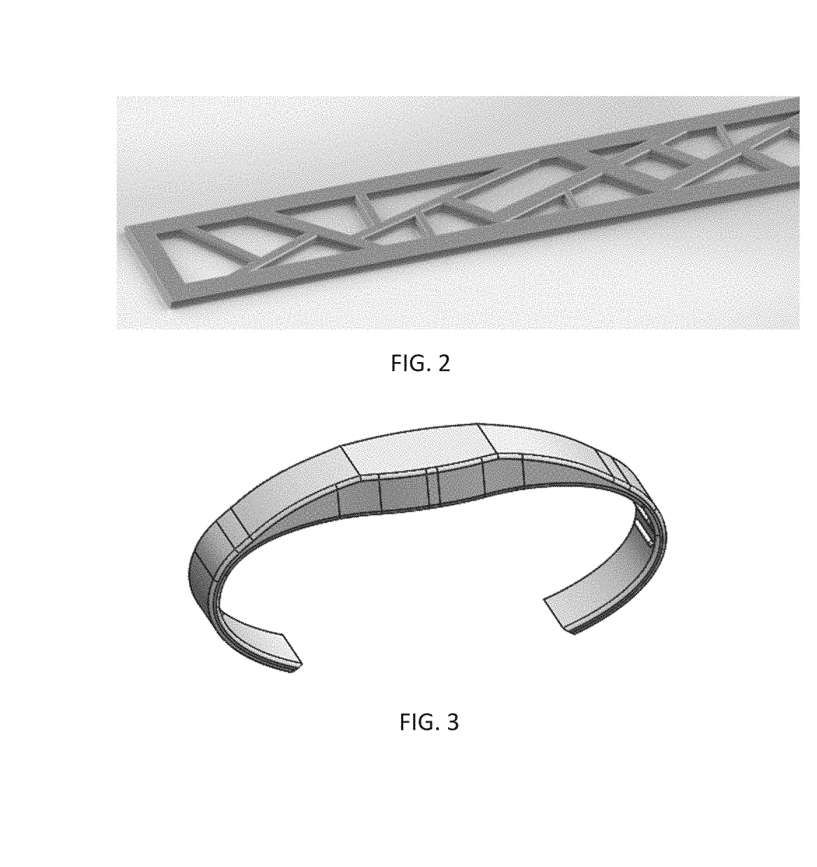

Various types of shapes/geometries or materials can be used to generate the linear strain/stress response. An example of another geometry would be a webbing pattern as shown in FIG. 2.

FIG. 3 illustrates an example of an apparatus having a semicircle band.

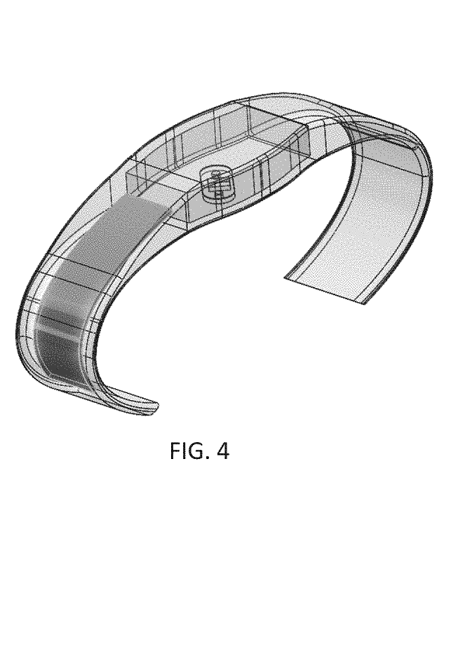

By using more rigid injection molded plastic or the placement of a rigid metal insert which has the desired shape, the band can act more like a bracelet that can wrap around the wrist and will not require it to be pulled over the fist, as shown in the example of FIG. 4. The rigid material will ensure the band is capable of applying adequate force on the wrist at the site where skin contact is required.

Any of the apparatuses described herein may include a bias (e.g., pneumatic/pressure bias, a mechanical, electrical or electromechaincal bias, a magnetic bias, a magnetoelecrical bias, etc.) that may selectively apply force to constrict the wrist band onto the subject to place one or more sensors (e.g., electrodes, etc.) in communication with the subject's skin at a predetermined pressure (e.g., an optimal pressure)+/−some range of values. A controller may be configured to regulate the force applied by the band, including the force applied by the bias to hold the sensor(s) against the skin. For example, the bias may be a solenoid.

In some variations the bias may be behind the sensor(s), driving them against the wearer's skin. Alternatively or additional, a bias may be on the apparatus opposite from the sensors. The controller may include a feedback loop that regulates the force applied by the bias based on the signal from the sensor. For example, the controller may apply force to hold one or more electrodes against the skin until a good electrical signal is received. If the signal is low or too irregular, the bias may increase (to a safety threshold) the pressure constricting the band and/or driving the electrode against a wrist worn in the band. The sensor may be optical, electrical, etc.

### User Interface

A simple user interface would allow the system to be used by a larger population of individuals. Complex interfaces will be difficult for people who have visual, cognitive, dexterous, or other issues typically present in the elderly population. The simplest interface is no interface. A simple interface can take the form of a band of LEDs along the length of the band that allows the entire band to be illuminated. This can be accomplished through a series of discrete LEDs that are exposed on the surface of the band, through one or more light pipes, through translucent material that allows the passage of light to the surface, or the entire band can be translucent allowing for the entire band to be illuminated. An RGB LED (or combination of LEDs) can be used to allow the band to glow different colors (or combinations of colors) in order to convey information in a simplistic fashion.

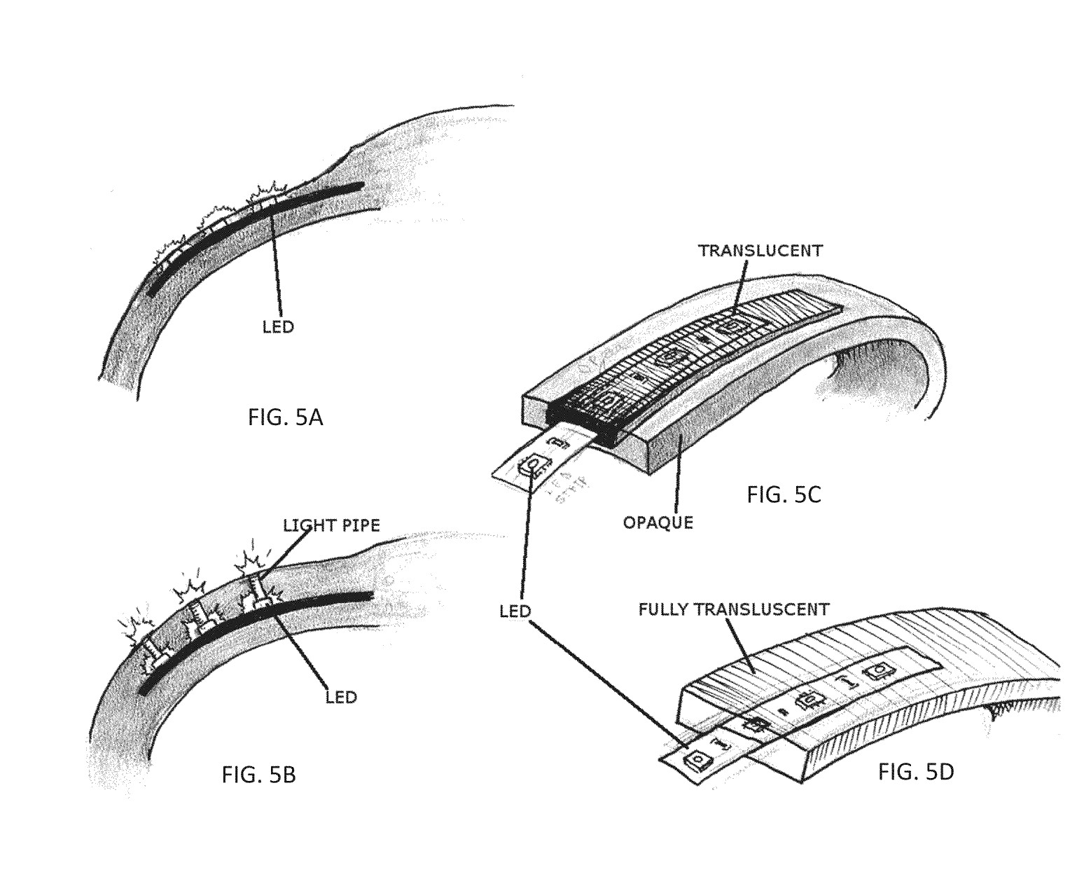

FIGS. 5A-5D illustrate a version of a band that is fabricated partially or fully with a translucent material and one or more LEDs (single color, combination of colors, or RGB) are embedded along the length of the strap. This can be accomplished by the use of a flexible circuit that runs along the length of the band. By illuminating the LEDs, the entire band (or portion thereof) will illuminate making it easy for the user to be notified of some event, such as a low battery. The use of different colors, combination of colors, or location of illumination can be used to convey different messages to the user. For example, if the band blinked red then the battery would be low. If the band blinked green then the band was fully charged.

Given that LEDs use current, it is desirable to ensure that the message is conveyed to the user at a time when the user is most likely to notice the notification. This can be accomplished by only displaying the notification when the user is awake. This can be accomplished by using an accelerometer or other activity monitor to measure when the user is active and not stationary—combined with the knowledge of the time of the day. For example, if the user shows activity from 8 AM-11 PM then this would be considered the time the user is awake. If there is limited activity after midnight, then it can be assumed that the user is moving in bed and showing some sort of notification would be wasteful of power. Finally, by showing the notification at the time of activity, then the chances the device will be noticed will be maximized. This can be accomplished by use of an accelerometer to detect motion.

### Battery & Noise (Smart Sensing)

Frequent recharging of the battery can result in the device running out of charge and no longer being able to relay data. For many physiologic processes, it is not important to continuously monitor the patients; rather, occasionally measuring the relevant physiologic parameters is sufficient. Additionally, for most physiologic measures, motion will cause the introduction of motion artifacts which will degrade the signal quality. By performing the measurement when the subject is stationary, the issue of motion artifacts can be averted and the quality the acquired signal will improve. Infrequent measurements will also greatly prolong the lifetime of the battery before the unit needs replacing or recharging.

Activity can be measured by the use of an accelerometer. However, most accelerometers will draw a certain level of current, which can impact the battery life. There exist ultra-low current accelerometers which can be configured to register acceleration beyond a certain threshold. These accelerometers will typically send a signal through an interrupt line to a microcontroller (or some other measuring device). The microcontroller can remain in a low power “sleep” state most of the time and only be awakened when the signal on the interrupt line changes. The microcontroller can then awaken, increment an internal counter, and resume its low power sleep state.

Inactivity can be determined if there is no increase in the counter value over a period of time. If motion occurs during the measurement, then the data can be discarded, the device can re-enter a sleep state, and wait for another period of inactivity before attempting to take a measurement.

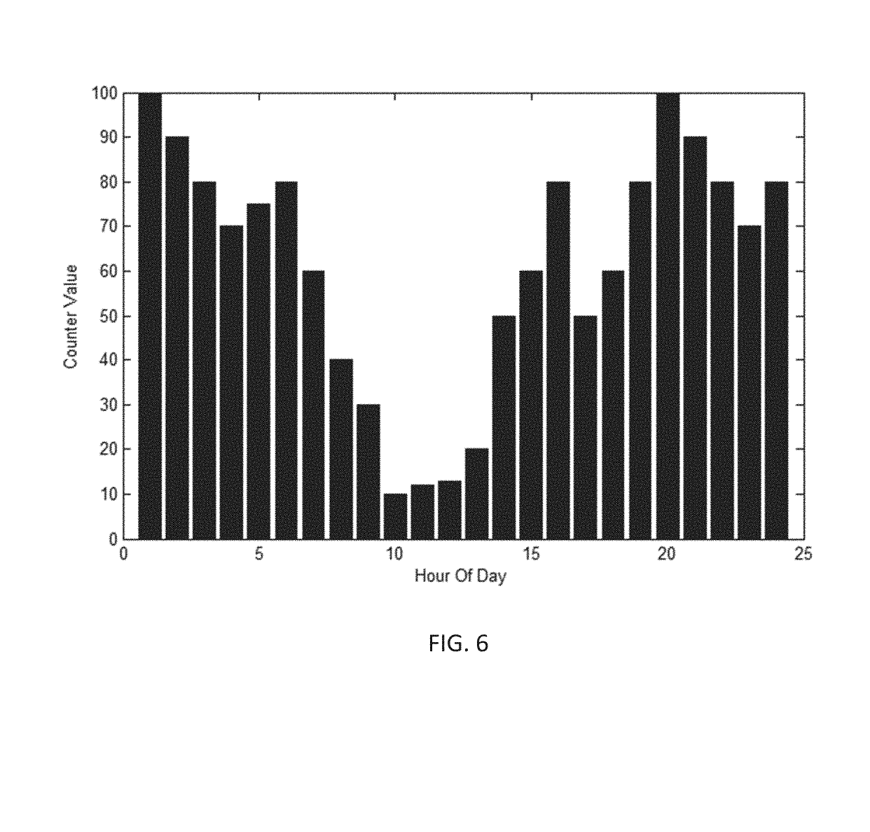

An individual's activity can be profiled by measuring the relative amount of activity throughout the day. This can be accomplished by recording the counter value over a period of time (e.g. 60 minutes), and examining the counter value throughout the day. FIG. 6 is a graph illustrating this. In this example, the individual was least active from 10:00 to 12:00. This window of time would represent the highest likelihood of making a measurement when the subject is stationary. By making measurements like this daily and computing the best time of day to make a measurement for each day of the week, an ideal time can be determined for the measurement per day of the week. This low period will most likely (but not necessarily) correspond to when the subject is sleeping.

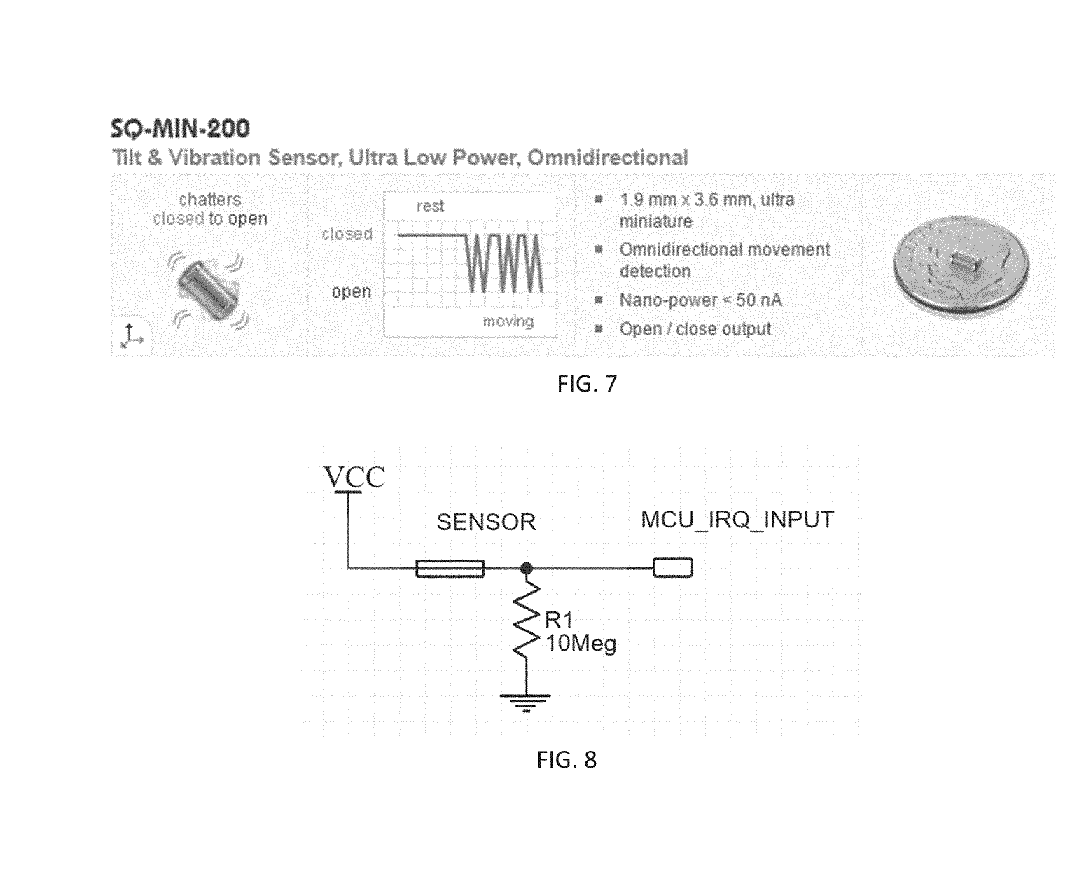

A lower-power option is to use an omnidirectional tilt and vibration sensor, such as the SignalQuest SQ-MIN-200. This device acts like a switch that opens and closes as the device moves. For example, see FIG. 7.

By configuring this device in series with a voltage source to an interrupt input on a microcontroller (FPGA or other similar device) with a pull-down on the input, it is possible to measure activity at a much lower power. When the switch is in an open state, the maximum current will be equal to the supply voltage divided by the value of the pull-down resistor. See, e.g., FIG. 8. The input to the microcontroller needs to be set to measure both high to low or low to high transitions for best accuracy, although either can be used as well.

### Recharge

In order to further simplify the user experience, the device can incorporate an inductive charging circuit. Incorporating a small antenna and a Qi charging control IC (http://www.qiwireless.com/) can enable such functionality. By using the embedded LEDs in the band to blink red (for example), the user can be notified that the battery is low. By removing the band and placing it on one of many commercially Qi charging pads, the user can avoid the need to directly connect a charging device to the band (i.e. with a USB cable). This is especially helpful for individuals who have difficulty with their hands and mobility. This will provide the added benefit of avoiding a connection which can allow water intrusion into the device by providing for a hermetically sealed unit.

### Telemetry

By incorporating a WiFi IC, such as the ESP8266, data can be relayed from the device to an access point, or an inexpensive cellular hotspot without the need of pairing to a smartphone, further increasing the simplicity of the system.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A wrist-worn heart monitor apparatus for monitoring a patient, the apparatus comprising:
a strap configured to be worn over the patient's wrist, the strap having an outer surface and an inner surface;
two more electrodes on the inner surface;
an activatable biasing element on the inner surface opposite the two or more electrodes;
a controller in the strap configured to activate and inactivate the bias to drive the two or more electrodes against the patient's wrist and to trigger sensing by the two or more electrodes; and
a motion sensor configured detect motion of the apparatus, wherein the controller is configured to trigger sensing only when the motion sensor does not sense motion.

2. The apparatus of claim 1, wherein the outer surface does not include any buttons or user-operated controls.

3. The apparatus of claim 1, wherein the bias comprises a mechanical bias.

4. The apparatus of claim 1, wherein the bias comprises an electromechanical bias.

5. The apparatus of claim 1, wherein the bias comprises a magnetic bias.
